(19) United States
(12) Patent Application Publication
(10) Pub. No.: US 2004/0152146 A1
(43) Pub. Date: Aug. 5, 2004

(54) Methods for screening compounds for use in the treatment of disease

(76) Inventors: William J. Wechter, Ojai, CA (US); E. David Murray JR., Redlands, CA (US); Elaine Benaksas Schwartz, Yorba Linda, CA (US); Israel Gutierrez, Colton, CA (US); David D. Quiggle, Yucaipa, CA (US)

Correspondence Address: HANA VERNY, PETERS, VERNY, JONES & SCHMITT, L.L.P., SUITE 230, 425 SHERMAN AVENUE, PALO ALTO, CA, 94306, US

(21) Appl. No.: 10/734,687

(22) Filed: Dec. 11, 2003

Related U.S. Application Data
(60) Provisional application No. 60/433,464, filed on Dec. 13, 2002.

(51) Int. Cl. C12Q 1/48

(52) U.S. Cl. 435/15

(57) ABSTRACT

Methods are disclosed for screening compounds for use in the treatment of, or the identification of a clinical or biological target for, a disease. The method comprises determining the ability of the compound to influence interactions involving alpha-methylacyl-CoA racemase.

This application claims priority over Provisional application Serial No. 60/433,464, filed Dec. 12, 2002.

### BACKGROUND OF THE INVENTION

Filed of the Invention

This invention relates to methods for screening compounds to ascertain the effectiveness of such compounds in the treatment of, or the identification of a clinical or biological target for, a disease state in a subject. The invention further relates to compounds effective in the treatment of a disease such as neoplastic disease, inflammation, inflammatory disease, pain, cystic fibrosis, dementia, and the like.

Neoplastic diseases are conditions in which abnormal proliferation of cells results in a mass of tissue called a neoplasm or tumor. Neoplasms have varying degrees of abnormalities in structure and behavior. Some neoplasms are benign while others are malignant or cancerous. An effective treatment intervention in the pathophysiologic progression of neoplastic disease would be considered a valuable contribution to the search for cancer preventive or curative procedures.

For example, the gastrointestinal tract, including the rectum and colon, is lined with epithelial cells, which have a high proliferation rate. The lining of the colon, in particular, made up of columnar rows of epithelial cells, is characterized by a series of indentations or crypts. Epithelial cells in the bottom regions of the crypts proliferate and move upward toward the tops of the crypts. In the normal colon, the proliferation region of the large intestine normally occupies the basal or deeper three-quarters of the crypts. A relationship has been observed between the expansion of cell proliferation zones to the upper regions of the crypts and colon cancer. See M. Lipkin, “Biomarkers of Increased Susceptibility to Gastrointestinal Cancer: New Application to Studies of Cancer Prevention in Human Subjects,” Cancer Research, Vol. 48, pp. 235-245 (Jan. 15, 1988).

Cancer of the colon is common in the western world and is an important cause of morbidity and mortality, having an incidence of about 5% in the U.S. population. As with other types of cancers, cancers of the gastrointestinal tract, including colon cancer, are characterized by development abnormalities in cell proliferation and differentiation in the gastrointestinal tract.

Another disease for which effective treatment is needed is cystic fibrosis. Cystic fibrosis (CF) is a heritable disease that follows an autosomal recessive pattern of transmittance. It is the most common lethal genetic disease in the United States. The approximate frequency in Caucasians is 1 in 2000. Cystic fibrosis is characterized by abnormal eccrine and exocrine gland function. In particular, mucous glands produce viscous secretions that lead to chronic pulmonary disease, insufficient pancreatic and digestive function and abnormally concentrated sweat.

The most prominent theories of CF etiology focus on alterations in physiochemical properties of exocrine secretions, the regulation of exocrine gland secretions, electrolyte transport and abnormalities in serum. Typical presentations include early onset of respiratory symptoms such as colds, and recurrent respiratory infections later in life. CF patients show evidence of decreasing pulmonary function with time, and their sputum cultures often display S. aureus, P. aeruginosa and P. capacia.

The major source of CF morbidity is pulmonary disease. More than 98% of CF patients die of either respiratory failure or pulmonary complications. Antibiotics are the key element in increasing survival. Prior to the 1950's, when modern antibiotics began to become available, patients typically survived for only a few years. At present, the median survival age is 32 years of age. Consequently, stimulation of neutrophil function as a means of clearing bacterial foci is thought to be an appropriate focus of treatment.

Still another disease for which effective treatment is needed is dementia including Alzheimer's Disease (AD), which is a degenerative brain disorder associated with extensive loss of specific neuronal subpopulations and characterized clinically by progressive loss of memory, cognition, reasoning, judgment and emotional stability that gradually leads to profound mental deterioration and ultimately death. AD is a common cause of progressive mental failure (dementia) in aged humans and is believed to represent the fourth most common medical cause of death in the United States. AD has been observed in varied races and ethnic groups worldwide and presents a major present and future public health problem. The disease is currently estimated to affect up to four million individuals in the United States alone. To date, AD has proven to be incurable, and presently causes up to 100,000 deaths yearly.

The brains of individuals with AD exhibit neuronal degeneration and characteristic lesions variously referred to as amyloidogenic plaques, vascular amyloid angiopathy, and neurofibrillary tangles. Large numbers of these lesions, particularly amyloidogenic plaques and neurofibrillary tangles, are generally found in several areas of the human brain. Smaller numbers of these lesions in a more restricted anatomical distribution are found in the brains of most aged humans who do not have clinical AD, as well as patients suffering from Down's Syndrome and Hereditary Cerebral Hemorrhage with Amyloidosis of the Dutch-Type.

Alpha-methylacyl-CoA racemase (AMACR) is a mitochondrial and peroxisomal enzyme that catalyzes the racemization of alpha-methyl, branched carboxylic coenzyme A thioesters. It is important in the oxidation of bile acid intermediates such as di- and trihydroxycholestanoic acid (DHCA and THCA) and branched chain fatty acids such as pristanic acid. AMACR catalyzes the conversion of several (2R)-methyl-branched chain fatty acyl-CoA's to their (S)-stereoisomers. Patients with a deficiency of AMACR accumulate in their plasma pristanic acid and the aforementioned bile acid intermediates. According to expressed sequence tag (EST) information, AMACR is expressed in most human tissues (uterus, kidney, brain, colon, prostate, lung, lymph node, connective tissue, pancreas). AMACR expression is consistently up-regulated in tumors, e.g., prostate, colon, breast, ovarian, colorectal, bladder, lung, renal, lymphoma and melanoma. Western Blot and immunohistochemical analysis confirms the up-regulation at the protein level and localizes the enzyme predominantly to the peroxisomal compartment of several tumors. A detailed immunohistochemical analysis of samples from prostate cancer cases using both standard slides and tissue microarray demonstrates that both prostate cancer and the High PIN lesions consistently score significantly higher than matching normal tissue.

A continuing need exists for compositions that are useful in the treatment of diseases and illnesses such as, by way of illustration and not limitation, neoplastic disease, inflammation, pain, cystic fibrosis, dementia, and the like. The compositions should be effective for preventing, delaying, and/or treating a disease, preferably, without some or all of the disadvantages of known treatments.

### SUMMARY OF THE INVENTION

One embodiment of the present invention is a method for screening a compound for use in the treatment of, or in the identification of a clinical or biological target for, a disease. The method comprises determining the ability of the compound to influence interactions involving alpha-methylacyl-CoA racemase.

Another embodiment of the present invention is a method for screening a small organic compound for use in the treatment (intervention in the pathophysiological process) of a disease. An analysis system is formed comprising the compound and alpha-methylacyl-CoA racemase. The analysis system is used under conditions for an interaction involving alpha-methylacyl-CoA racemase to occur. The amount or activity of alpha-methylacyl-CoA racemase in the system is measured and related to the effectiveness of the compound in the treatment of a disease or illness.

Another embodiment of the present invention is a method for screening a small organic compound for use in the treatment of a disease. An analysis system is formed comprising the compound, alpha-methylacyl-CoA racemase and a ligand for alpha-methylacyl-CoA racemase. The analysis system is used under conditions for an interaction between the ligand and the alpha-methylacyl-CoA racemase to occur. The amount or activity of alpha-methylacyl-CoA racemase in the system is measured and related to the effectiveness of the compound in the treatment of a disease or illness.

Another embodiment of the present invention is a method for screening a small organic compound for use in the treatment of a disease. An analysis system is formed comprising the compound and a host that expresses alpha-methylacyl-CoA racemase. The analysis system is used under conditions for an interaction involving alpha-methylacyl-CoA racemase to occur. The amount or activity of alpha-methylacyl-CoA racemase in the system is measured and related to the effectiveness of the compound in the treatment of a disease.

Another embodiment of the present invention is a method for preventing, delaying and/or treating a disease. The method comprises administering to a subject with the disease a pharmaceutically effective a mount of a compound ascertained by one of the aforementioned methods.

### DESCRIPTION OF THE SPECIFIC EMBODIMENTS

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

The present inventors have discovered that interactions involving AMACR surprisingly provide an exquisite analytical system for screening compounds for use in the treatment of a disease. Compounds are screened and selected based on their ability to influence AMACR activity or production. The compounds selected in the above manner are effective in the prevention, delaying and/or treatment of a disease state. In many instances the area of effectiveness of the selected compounds can be predicted if an association of AMACR activity and/or production with a particular disease state is known. This invention describes a method of screening for disease and compounds that will intervene in the disease based on the detection of specific/selected products of AMACR activity and amounts of AMACR.

A disease, in general, is any departure from a state of health; an illness or a sickness; an unhealthy condition of an animal or a part thereof including an unhealthy condition, which may be caused by, or incident to, therapy applied to such animal.

A disease related to AMACR function is a disease state for which an association with the function of AMACR has been made. For example, over-expression of AMACR has been associated with, for example, prostate cancer, colon cancer, breast cancer, ovarian cancer, colorectal cancer, bladder cancer, lung cancer, renal cancer, lymphoma and melanoma. Other disease states that may be treated with a compound identified in accordance with the present invention include, by way of illustration and not limitation, inflammation, cystic fibrosis, dementia, neoplastic disease, pain, and so forth. Dementia includes Alzheimer's disease, and so forth. Neoplastic disease includes cancers such as, for example, those cancers mentioned above, and cervical cancer, endometrial cancer, testicular cancer, pancreatic cancer, leukemia, squamous cell carcinoma, lipoma, brain tumors, and the like.

Treatment of a disease involves the administration of a compound identified in accordance with the present methods to a subject in need thereof to bring about a preventative, alterative, modulatory, therapeutic or curative effect. Thus, the term “treatment” or “treating” includes preventing the disease from occurring where no symptoms are yet displayed, inhibiting the onset, or arresting the development of, a disease, relieving the disease, i.e., causing the regression of the disease, or prolonging subject survival, altering progression of the disease, modulating the disease, and the like.

Identification of a clinical target for a disease means identification of the modification, modulation of a sign, or the like, and/or symptom, or the like, etc., associated with a disease state wherein molecules administered to a patient produce a change in a sign, symptom, or disease outcome associated with a disease state that may be useful in the identification of molecules for, or useful in the prevention and/or treatment, of a disease.

Identification of a biological target for a disease means the identification of a receptor or the like, biomarker, or the like, clinical laboratory measurement or the like, etc., wherein molecules that bind thereto or are expressed thereat may be useful in the identification of molecules for, or useful in the treatment of, a disease.

A compound that is subjected to screening by the methods of the present invention may be any compound of interest and includes small organic compounds, proteins, peptides, higher molecular weight carbohydrates, polynucleotides, fatty acids and lipids, and the like. Compounds may be screened individually or in sets or combinatorial libraries of compounds may be screened based on the methods disclosed herein. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, acidification, etc. to produce structural analogs.

In screening using a combinatorial library, a large library of chemically similar or diverse molecules are screened for the desired biological activity. This approach has become an effective and hence important tool for discovery of new drugs. In one approach combinatorial synthesis is employed to prepare a diverse set of molecules in which several components predicted to be associated with the desired biological activity are systematically varied. In combinatorial screening, the number of hits discovered is proportional to the number of molecules tested. The large numbers of compounds, which may reach thousands of compounds tested per day, are screened using one of the assay approaches of the invention and a suitable high throughput screening technique, in which laboratory automation and robotics may be applied.

A small organic compound, as the term is used herein, is a compound of molecular weight less than about 5000, usually less than about 2500, usually, less than about 2000, more usually, less than about 1500, preferably about 100 to about 1000, more preferably about 300 to about 600. The compound should be pharmaceutically acceptable, i.e., able to be administered to a subject at levels that are not toxic to such subject or capable of being rendered pharmaceutically acceptable such as by conversion to a salt, placed in a suspension, dissolved in a solution such as to make it bioavailable to the sites/locations, anatomic and inter- and intracellular, of AMACR and so forth. Pharmaceutically acceptable means that the compound is useful in preparing a pharmaceutical composition that is generally safe, non-toxic and neither biologically nor otherwise undesirable.

As indicated above, the small organic compounds may be either biological or synthetic organic compounds. The atoms present in the small organic compound are generally in the group comprising carbon, hydrogen, oxygen, and nitrogen and may include phosphorus and sulfur if in a pharmaceutically acceptable form. Generally, oxygen, nitrogen, sulfur or phosphorus, if present, are bound to carbon or one or more of each other or to hydrogen to form various functional groups such as, for example, carboxylic acids, alcohols, thiols, carboxamides, carbamates, carboxylic acid esters, amides, ethers, thioethers, thioesters, phosphates, phosphonates, olefins, ketones, amines, aldehydes, and the like. The small organic compounds, as the term is used herein, also include small peptides, small oligonucleotides, small polysaccharides, fatty acids, lipids, and the like having a molecular weight less than about 5000.

Small organic compounds are desirable for treatment of disease for several reasons related to drug delivery. Because of their small size they are more likely to be permeable to cells. Unlike peptides or oligonucleotides, they are less susceptible to degradation by many cellular mechanisms. They are not as apt to elicit an immune response. Many institutions have extensive libraries of chemical and/or biological compounds and mixtures, often fungal, bacterial, or algal extracts that would be desirable to screen with an assay such as that of the present invention.

Proteins are poly(amino acids) that have a molecular weight of at least about 5,000, more usually at least about 10,000. The poly(amino acids) of interest will generally be from about 5,000 to about 5,000,000 or more molecular weight, more usually from about 20,000 to about 1,000,000 molecular weight. A wide variety of proteins may be considered such as a family of proteins having similar structural features, proteins having particular biological functions, proteins related to specific microorganisms, particularly disease causing microorganisms, etc. Such proteins include, by way of illustration and not limitation, cytokines or interleukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, unclassified proteins, e.g., somatotropin, prolactin, insulin, pepsin, proteins found in human plasma, blood clotting factors, blood typing factors, protein hormones, cancer antigens, tissue specific antigens, peptide hormones, nutritional markers, tissue specific antigens, and synthetic peptides, which may or may not be glycated.

As used herein, small peptides are poly(amino acids) that have a molecular weight less than about 5000.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The higher molecular weight polynucleotides can have from about 20 to about 5,000,000 or more nucleotides. Polynucleotides include single or multiple stranded configurations, where one or more of the strands may or may not be completely aligned with another.

As used herein, small oligonucleotides are those polynucleotides having a molecular weight of less than about 5000. The small oligonucleotides have at least about 2 nucleotides, usually, about 5 to about 20 nucleotides. Higher molecular weight carbohydrates are complex polyalcohols having a molecular weight greater than about 5000. They include polysaccharides, which may be associated with a protein or cell surface such as a proteoglycan, glycoprotein, and the like. Examples of carbohydrates include, by way of illustration and not limitation, monosaccharides, oligosaccharides, polysaccharides, cyclodextrins, heparin sulfate and the like.

Interactions involving AMACR include, for example, the ability of a ligand for AMACR to interact with AMACR, the expression of AMACR, or inhibition of production of AMACR, in living systems such as animals, plants, animal or plant tissue or cells, transgenic cell lines, transgenic animals, chemically or genetically altered tissue or cells, and so forth. Samples, as used herein, include biological fluids such as blood, plasma, serum and the like, organ or tissue or cell culture derived fluids; and fluids extracted from physiological tissues. Also included in the term are derivatives and fractions of such fluids. The cells may be dissociated, in the case of solid tissues, or tissue sections may be analyzed. Alternatively, a lysate of the cells may be prepared for the purpose of a screening assay in accordance with the present invention. Cell homogenates, differential centrifugation, cellular fractions/extracts, differentiation through the use of protease inhibitors, immunoblotting and enzyme assays and the like may also be employed.

The compound may influence the interactions involving AMACR in a number of ways depending on the nature of the interaction. The compound may modulate the activity of AMACR or the production of AMACR, or modulate the products of AMACR, or the accumulation of substrate and so forth. The ability of a compound of interest to influence an interaction involving AMACR may be made by a variety of determinations depending on the interaction involving AMACR. The following discussion and examples are by way of example and not limitation. The concepts of the present invention may be applied to any interactions involving AMACR whether now known or discovered in the future.

One approach involves determining the amount or activity of AMACR resulting from the ability of the compound of interest to influence the interaction of AMACR with a ligand for AMACR and relating the amount or activity thereof to the effectiveness of the compound in the treatment of, or in the identification of a clinical or biological target for, a disease. A ligand for AMACR is a molecule to which AMACR can bind. Usually, the binding is specific, which involves the specific recognition of one of two different molecules for the other compared to substantially less recognition of other molecules. The ligand for AMACR may be a substrate for AMACR. As mentioned above, AMACR is a mitochondrial, peroxisomal and microsomal enzyme that catalyzes the racemization of alpha-methyl, branched carboxylic coenzyme A (CoA) thioesters. Other ligands include, for example, aryl-propionic acids, benzoates, fatty acids, acyl-CoA esters, and thiazolidinedione analogs.

Thus, one group of ligands for AMACR, for example, includes CoA thioesters of alpha-methyl acyl fatty acids where the alpha carbon is an R-stereoisomer. Usually, the fatty acids are organic monobasic acids that comprise a carboxylic acid moiety and a hydrocarbon moiety having about 1 to about 50 or more carbon atoms, usually, about 2 to about 30 or more carbon atoms where the carbon alpha to the carboxylic acid moiety has an alpha methyl group. The compounds may be branched or unbranched, saturated (0 unsaturations) or unsaturated. The unsaturated fatty acid compounds may comprise from 1 to about 10, usually, 1 to about 5, unsaturations, which may be double bonds, triple bonds or a combination of both. Generally, the hydrocarbon moiety may or may not comprise cyclic moieties or rings. Carbon atoms of the hydrocarbon moiety may comprise one or more substituents such as, for example, alkyl, alkenyl, alkynyl, and the like. Usually, the number of substituents present in the fatty acid compounds other than alkyl is 1 to about 10, more usually, 1 to about 5. “Alkyl” means a branched or unbranched saturated monovalent hydrocarbon radical containing 1 to about 30 or more carbon atoms, such as methyl, ethyl, propyl, tert-butyl, n-hexyl, iso-hexyl, n-octyl, iso-octyl, and so forth. Alkyl includes lower alkyl. “Lower alkyl” means a branched or unbranched saturated monovalent hydrocarbon radical containing 1 to about 10 carbon atoms, such as methyl, ethyl, propyl, isopropyl, tert-butyl, iso-butyl, n-pentyl, iso-pentyl, and so forth. Preferably, the alkyl substituent is lower alkyl, more preferably, methyl. “Alkenyl” means a branched or unbranched unsaturated hydrocarbon radical containing at least one double or ethenylic bond and 2 to 30 or more carbon atoms and includes lower alkene, unless otherwise indicated. “Lower alkenyl” means a branched or unbranched unsaturated hydrocarbon radical containing at least one double or ethenylic bond and 2 to 6 carbon atoms, unless otherwise indicated. “Alkynyl” means a branched or unbranched unsaturated hydrocarbon radical containing at least one triple or ethynylic bond and 2 to 30 or more carbon atoms and includes lower alkyne, unless otherwise indicated. “Lower alkynyl” means a branched or unbranched unsaturated hydrocarbon radical containing at least one triple or ethynylic bond and 2 to 6 carbon atoms, unless otherwise indicated. One or more carbon atoms of the hydrocarbon are optionally substituted.

The CoA thioesters of the fatty acid compound may be formed in situ or may be preformed. Where the CoA thioester is formed in situ, the analytical system includes reagent(s) for forming the CoA thioester. Such reagents include, for example, a fatty acid compound, an enzyme that facilitates formation of the CoA thioester of the fatty acid compound such as, e.g., acyl-CoA synthetase, AMP-forming, EC 6.2.1.3, fatty acid CoA ligase and the like, CoA-SH, ATP, GTP, Mg²⁺, buffers, media and so forth.

Another in situ approach involves employment of a cell extract, lysate, subfraction or such that would reasonably be expected to contain an enzyme such as acyl-CoA synthetase plus the necessary reagents, the fatty acid compound, CoA-SH, ATP, GTP, Mg²⁺, buffers, media and so forth.

The ligand may be preformed by organic synthesis and the like. In one example in accordance with this embodiment, the CoA thioesters may be formed by mixing the proper reagents such as a fatty acid compound, or activated derivative thereof such as an anhydride or the like, CoA-SH, and any other chemicals and so forth necessary to produce the thioester in an appropriate solvent for the reaction. The above are mixed under conditions so that upon completion of the reaction and any necessary steps to yield the ligand, the ligand can then be mixed with the analysis system.

The ligand for AMACR may be an endogenous ligand, i.e., a ligand found naturally in, or whose presence is induced in, the tissue of a mammalian subject or plant and is, thus, a biological ligand. Usually, the endogenous ligand is associated with a cell such as, for example, liver, kidney, pancreas, brain, monoblastic cell lines, yeast and plant cells and so forth. As mentioned above, AMACR is important in the oxidation of bile acid intermediates such as di- and trihydroxycholestanoic acid (DHCA and THCA) and branched chain fatty acids such as pristanic acid.

In one embodiment of the present invention an analytical system is formed comprising an assay medium, the compound of interest, AMACR, a ligand for AMACR or a precursor thereof, and any ancillary agents as required. Such ancillary agents depend on the nature of the assay and may include one or more of, for example, salts, neutral proteins, e.g. albumin, detergents, etc., that are used to facilitate optimal protein-protein or protein-ligand binding and/or reduce non-specific or background interactions, reagents that improve the efficiency of the assay, such as protease inhibitors, nuclease inhibitors, anti-microbial agents, anti-fungal agents, controls, thioesterase inhibitors, etc.

The AMACR may be from any source, natural or synthetic, which is convenient for the assay. Accordingly, the AMACR may be isolated from a source of the enzyme such as, for example, cytosol, mitochondria, peroxisomes, microsomes, and ER (endoplasmic reticulum) and the like. In a particular example, AMACR may be isolated from rat liver and kidney cytosol and mitochondria by one or more known techniques that include centrifugation, dialysis, gel filtration chromatography, ammonium sulfate precipitation, chromatography such as, e.g., DEAE-sepharose chromatography, hydroxyl apatite chromatography, phenyl-sepharose CL-4B chromatography, red dye affinity chromatography, sephacryl S-200 chromatography, and so forth. See, for example, Shieh, et al., J. Biol. Chem. 1993. 268(5):3487-3493. Other approaches include ion exchange chromatography on other solid phases, other affinity chromatography media, other size-exclusion chromatography media.

The AMACR may also be obtained using recombinant techniques (see, for example, Amery, et al., J. Lipid Res. 2000 41:1752-1759) and Kotti, et al., J. Biol. Chem. (2000) 275(27):20887-20895. Other sources of AMACR include chemical synthesis of the protein or use of cDNA or a synthetic oligonucleotide encoding the sequence of AMACR to produce the protein in an expression system and so forth.

An expression system used in the present invention may be any suitable expression system. Examples include, but are not limited to, insect cell expression systems, mammalian cell expression systems, yeast cell expression systems, bacterial expression systems, plant expression systems, and so forth.

Mammals or mammalian are used broadly to describe organisms that are within the class mammalia, including the orders carnivore (e.g., dogs and cats), rodentia (e.g. mice, guinea pigs, and rats), and primates (e.g., humans, chimpanzees, and monkeys).

The specific activity of the AMACR used in the above assay is usually about 0.1 to about 500 Units per mg protein. The lower number for systems such as cell lysates and the upper number for purified AMACR. Unit is μmole of S-2-(4-isobutylphenyl)propionic acid CoA ester converted to R in one minute at 30° C. (Shieh, supra)

The assay medium is usually an aqueous medium, but other polar solvents may be employed. These other solvents are usually oxygenated organic solvents of from 1-6, more usually from 1-4, carbon atoms, including alcohols, ethers and the like. These cosolvents may be present in less than about 70 weight percent, more usually, in less than about 30 weight percent.

The pH for the medium is usually in the range of about 4.5 to 9.5, more usually in the range of about 5.5-8.5, and preferably in the range of about 6-8. The pH and the temperature are chosen and varied as the case may be to maximize the ability of the compound to be screened to influence the interaction of AMACR with its ligand. Various buffers may be used to achieve the desired pH and maintain the pH during the assay. Illustrative buffers include borate, phosphate such as phosphate buffered saline and the like, carbonate, Tris, barbital, HEPES, MOPS, and the like. The particular buffer employed is not critical to the invention but in individual methods one buffer may be preferred over another.

In addition to buffers, the medium can contain other agents such as stabilizers for the assay medium or assay components such as antibiotics, protease inhibitors, nuclease inhibitors, anti-microbial agents, anti-fungal agents, thioesterase inhibitors, pH indicators and the like, serum proteins such as albumins, etc., and the like. Normally, such agents are present in amounts effective to achieve the desired result such as stabilization, pH control, and so forth; the appropriate amounts are well known to the skilled artisan.

Moderate temperatures are normally employed for carrying out the method. In general the temperature should be that which promotes interaction of AMACR with the ligand for AMACR and promotes the influence on that interaction by the screened compound if such influence is to occur. The temperature should not be so great as to be detrimental to the reagents used in the assay. The temperatures for the method are generally in the range of from about 0° C. to a bout 95° C., more usually from about 10° C. to about 50° C., and preferably, about 20° C. to about 40° C.

The time period for the incubation in accordance with the present method is generally long enough to permit interaction of AMACR with its ligand and to permit the compound to influence this interaction if such compound is able to do so. Generally, the time period for incubation is about 1 to about 60 minutes, usually, about 2 to about 30 minutes, and preferably, about 5 to about 10 minutes.

The amount employed of the compound to be screened, of AMACR and of ligand for AMACR may vary depending on the type of assay, the nature of the compound screened, the activity of AMACR, the purity of the AMACR, the nature (composition) of the incubation medium the source of AMACR and so forth. The amount of the compound tested in the assay is usually about 0.1 to about 2500 μg per milliliter (ml) of assay medium, preferably, about 1 to about 500 μg per ml of assay medium, and more preferably, about 10 to 30 μg per ml of assay medium. It is preferable to test the compound at a number of different concentrations. In general, the assay medium contains AMACR at an appropriate concentration, usually, about 0.01 to about 2 Units per ml, preferably about 0.1 to about 1 Unit per ml. In general, the assay medium contains ligand for AMACR at an appropriate concentration, usually, about 0.1 to about 10,000 μg per ml, preferably about 10 to about 500 μg per ml. The amounts of compound screened and ligand can also be expressed relative to the amount (activity) of AMACR. In this manner the compound tested in the assay is usually about 0.05 to about 250,000 μg per Unit of AMACR activity, preferably, about 1 to about 5000 μg per Unit of AMACR activity, and more preferably, about 10 to 300 μg per Unit of AMACR activity. Additionally, in this manner the ligand for AMACR at an appropriate concentration, usually, about 0.05 to about 1,000,000 μg Unit of AMACR activity, preferably about 10 to about 5000 μg Unit of AMACR activity. It should be understood that in some instances, particularly with regard to crude cell preparations, the amount (mg protein), activity and specific activity (enzyme activity per amount of protein) of the AMACR may vary greatly and in some systems may not be known. With these preparations the above amounts are determined empirically.

The above screening methods may be designed in a number of different ways, where a variety of assay configurations and protocols may be employed, as are known in the art. All of the reagents may be in solution phase or one of the reagents may be bound to a solid support and the remaining reagents contacted with the support bound reagent. The above reagents of the method may be combined at substantially the same time or at different times.

After the appropriate incubation period, the reaction may be terminated by addition of a terminating agent such as, e.g., hydroxylamine, trichloroacetic acid, and the like. The extent that the compound of interest has influenced the interaction between AMACR and its ligand is determined. To this end, one may determine the amount or activity of AMACR, the production, or inhibition of production, of product from the ligand, the amount of ligand remaining, and so forth. The CoA ester products may be analyzed directly or they may be hydrolyzed by a suitable hydrolysis agent such as, e.g., dilute mineral acid (hydrochloric acid, sulfuric acid, phosphoric acid, and so forth. The resulting 2-(4-isobutylphenyl)propionic acid may be activated such as by formation of an acyl halide (acyl chloride, acyl bromide, etc.) and converted to an ester such as, e.g. menthyl ester.

In the aforementioned assay method, one convenient approach is to determine the amount of product from the ligand or the amount of ligand remaining. The determination depends on the nature of the ligand and/or the nature of the product. Where the ligand is a CoA thioester of an alpha-methyl acyl fatty acid having the alpha carbon as an R-stereoisomer, the extent of racemization of the R-stereoisomer may be determined. To this end, standard techniques may be employed such as, for example, directing a beam of polarized light from a polarized light source towards the assay medium contained in a suitable container such as, e.g., polarimeter cuvette, and monitoring the transmitted light with a polarized light detector. The composition may be analyzed by HPLC and the results compared to a control assay without the AMACR from the aforementioned assay method. Other approaches include analysis by chiral HPLC, a method by which enantiomers can be resolved and quantitated.

AMACR activity may be measured in a variety of ways. In one approach an enantioselective HPLC method may be employed. To this end, the AMACR present after the aforementioned incubation may be combined with a substrate for AMACR such as, for example, (R)-2-(4-isobutylphenyl)propionyl-CoA. The reaction may be terminated by addition of a terminating a gent such as, e.g., hydroxylamine, trichloroacetic acid and the like. The CoA ester products may be hydrolyzed by a suitable hydrolysis agent such as, e.g., dilute mineral acid, such as hydrochloric acid, or dilute base, such as sodium hydroxide and so forth. The resulting 2-(4-isobutylphenyl)propionic acid may be activated such as by formation of an acyl halide (acyl chloride, acyl bromide, etc.) and converted to an ester such as, e.g. menthyl ester. The composition is then analyzed by HPLC and the results compared to a control assay without the AMACR from the aforementioned assay method. See, for example, Shieh, et al., supra. Alternatively, the degree of inversion of the (R)-2-(4-isobutylphenyl)propionyl-CoA may be estimated as the amount of S-enantiomer formed during incubation (see, for example, Reichel, et al., Biochem. Pharmacol. 1995 50(11):1803-1806) (Reichel 1).

In another approach an antibody is employed that is capable of binding to AMACR. The antibody is combined with the assay medium and a determination is made as to the level of binding of the antibody to the AMACR. The amount of antibody that binds is directly related to the amount of AMACR such as, for example, the amount of expression, or prevention of expression, of AMACR resulting from the presence of the compound being screened.

The antibody can be monoclonal or polyclonal and can be prepared by techniques that are well known in the art such as immunization of a host and collection of sera (polyclonal), by preparing continuous hybrid cell lines and collecting the secreted protein (monoclonal) (see, e.g., Kohler and Milstein, Nature (1975) 265:495-497) or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b, and IgG3, IgM, etc. Fragments thereof may include Fab, Fv and F(ab′)₂ Gab′, and the like. In addition, aggregates, polymers, and conjugates of immunoglobulins or their fragments can be used where appropriate so long as binding affinity for a particular molecule is maintained. The antibody may be a polyclonal antibody such as described by Reichel 1.

In one approach the antibody is labeled with a reporter molecule and the signal from the reporter molecule is determined and related to the amount of AMACR in the medium. Alternatively, the antibody does not contain a reporter molecule. A second antibody is employed that is specific for the first antibody where the second antibody has a reporter molecule. The first antibody may be unbound to or may be bound to a surface or support. After incubation of the assay medium as above, the medium is combined with the support, if employed, to capture any AMACR. Next, the medium is removed from the support, which is usually washed and then the second antibody containing the reporter molecule is contacted with the support. Alternatively, the second antibody can be added to the medium that was separated from the support. In either approach the signal from the reporter molecule is determined and related to the amount of AMACR, which in turn is related to the effectiveness of the compound of interest against a disease. The values obtained by the detection method employed are usually compared to a control value to determine any relative differences, where the difference may be measured qualitatively and/or quantitatively. The differences are then related to the extent that the compound of interest has influenced the interaction involving AMACR. The use of controls is well known in the art and will not be discussed in detail herein.

The reporter molecule or label is a chemical entity capable of being detected by a suitable detection means, including, but not limited to, spectrophotometric, chemiluminescent, immunochemical, or radiochemical means. The reporter molecule can be conjugated to another molecule such as an antibody by procedures well known in the art. Reporter molecules are members of a signal producing system capable of being detected directly or through a specific binding reaction to produce a detectable signal. The reporter molecule can be isotopic or nonisotopic, usually nonisotopic, and can be a catalyst, dye, fluorescent molecule, chemiluminescent molecule, coenzyme, enzyme, substrate, radioactive group, certain particles such as carbon, gold, and the like. The signal producing system includes all of the reagents required to produce a measurable signal. Other components of the signal producing system can include substrates, coenzymes, enhancers, activators, chemiluminescent compounds, cofactors, inhibitors, scavengers, specific binding substances, and the like.

Another embodiment of a screening assay in accordance with the present invention involves tissue such as cells that comprise AMACR, which may be employed to screen compounds of interest in accordance with the present invention. The primary components of a screening assay in accordance with this embodiment of the present invention are the compound to be screened and cells having AMACR. A cell culture, cell dispersion/suspension, obtained by perfusion or digestion of a tissue with proteases and/or collagenase or the like, containing the tissue or cells comprising AMACR is prepared in accordance with known procedures. The cells from the culture are generally washed with an appropriate buffer prior to use in an assay. The cells in the buffered medium are then incubated with the compound to be screened to form an assay medium. The medium is usually an aqueous medium having a pH as discussed above. In addition to buffers, the medium can contain other agents such as stabilizers for the assay medium or assay components such as antibiotics and the like, serum proteins such as albumins and the like. Normally, such agents are present in amounts effective to achieve the desired result such as stabilization, pH control, and so forth; the appropriate amounts are well known to the skilled artisan. The temperature, times for incubation, and so forth are also discussed above and apply to the assay employing cells. The aforementioned discussion applies as well to cell lysates.

The amount of the compound tested in the assay is usually about 0.1 to about 2500 μg per ml of assay medium, preferably, about 1 to about 500 μg per milliliter (ml) of assay medium, and more preferably, about 10 to 30 μg per ml of assay medium. It is preferable to test the compound at a number of different concentrations. In general, the assay medium contains the appropriate cells at a concentration of about 10² to about 10⁹ cells per ml, preferably about 10³ to about 10⁸ cells per ml, more preferably, about 10⁴ to about 10⁶ cells per ml. The amount of the compound to be screened and the number of cells employed may vary depending on the type of assay employed and the nature of the compound screened.

After the cells have been incubated with the compound, an examination is made to determine the influence of the compound of interest on the interactions involving AMACR in the cell system. This examination may be carried out in a number of different ways.

In one approach an antibody is employed that is capable of binding to AMACR. The aforementioned discussion of the use of antibodies in the detection step of the present methods applies equally to this assay embodiment.

In another approach the amount of AMACR may be determined by DNA microarray analysis to measure AMACR gene expression. Using AMACR-specific primers, reverse transcriptase-polymerase chain reaction (RT-PCR) (see, for example, Rubin, et al., JAMA (2002) 287(13):1662-1670) may be performed on the medium such as, e.g., tissue samples, cells, cell homogenates, cell lysates, or sub-cellular fraction tissue homogenates from the above screening assay. In a particular example, the sample may be tissue in which over-expression of AMACR occurs such as, for example, tissue from individuals having prostate cancer. These samples may be combined with the compound of interest and the screening assay in accordance with the invention may be carried out as described above. Determination of AMACR may be carried out by the above-mentioned DNA microarray analysis. For additional discussion of DNA microarray analysis of AMACR, see Luo, et al., Cancer Research (2002) 62:2220-2226 and Reichel, et al., Molecular Pharmacol. (1997) 51:576-582 (Reichel 2).

In another approach the amount of AMACR may be determined by Northern blot analysis of poly(A) and RNA, particularly, from tissue and cells. Briefly, mRNA is isolated from tissue or cells and hybridized with cAMACR as a probe and the signal is detected and related to the amount of AMACR in the tissue or cells. See, for example, Kotti, et al., supra, and Reichel 2. Alternatively, Southern blotting and hybridization techniques may be used. See, for example, Kotti, et al., supra.

Immunoelectron microscopy may be employed to determine levels of AMACR in the samples from the screening assays in accordance with the invention. Samples may be exposed to polyclonal AMACR antibody and antibody distribution may be identified by gold particles conjugated to protein A. See, for example, Kotti, et al., supra.

In another approach immunoisolation of AMACR may be employed. To this end, samples from the screening assay in accordance with the present invention may be subjected to immunoaffinity chromatography using a column comprising antibodies for AMACR. Activity of AMACR may be determined using [2-³H]pristanoyl-CoA and [24,25-³H]THCA-CoA. See, for example, Kotti, et al., supra.

In another approach the amount of AMACR may be determined by chromosomal localization. See, for example, Kotti, et al., supra.

Immunoblot analysis, e.g., Western blot analysis, may be employed to determine the amount of AMACR in the aforementioned screening assays. Samples may be mixed with sodium dodecyl sulfate sample buffer and electrophoresed onto a sodium dodecyl sulfate-polyacrylamide gel under reducing conditions. The separated proteins are transferred to nitrocellulose membranes and incubated. Antibodies to AMACR with a suitable detectable label are applied and signal level is determined and related to the amount of AMACR in the samples. See, for example, Kotti, et al., supra, Rubin, et al., supra, and Luo, et al., supra.

In another approach the amount of AMACR may be determined by cDNA PCR (Polymerase chain reaction) amplification (molecular cloning and sequencing). Briefly, AMACR mRNA from the aforementioned screening assay in accordance with the present invention is amplified using PCR. The AMACR mRNA from the screening assay is combined with appropriate PCR primers and other PCR reagents such as a polymerase and treated under standard PCR conditions. The products from the PCR amplification may be purified and PCR amplification repeated. More specifically, the amount of AMACR mRNA can be determined by the use of quantitative RT-PCR, which is an automated methodology of the aforementioned process. The number of cycles required to reach a certain number of cDNA copies indicates the number of copies of mRNA present in the original incubation.

Purified PCR products may be cloned and transformed in accordance with standard practice with resulting expression of recombinant AMACR from the transformants. Sequencing may be carried out according to standard techniques. See, for example, Kotti, et al., supra, Luo, et al., supra, and Reichel 2. In another approach sequence data analysis may be employed to determine the amount of AMACR from the screening assay. See, for example, Kotti, et al., supra, and Reichel 1).

Another approach to analyzing the results of the screening assay involves gas-liquid chromatography-mass spectrometry (GC-MS) analysis of phytanic acid, pristanic acid and trimethylundecanoic acid (THA) isomers particularly in plasma. Phytanic acid is a branched chain fatty acid derived from dietary sources and broken down in the peroxisome to pristanic acid via alpha-oxidation. Pristanic acid then undergoes beta-oxidation in peroxisomes. Phytanic acid naturally occurs as a mixture of diastereomers at the 2-position. In contrast to the alpha-oxidation system, peroxisomal beta-oxidation is stereospecific and only accepts (2S)-isomers. AMACR is required to convert (2R)-pristanic acid as the CoA ester to its (2S)isomer as the CoA ester.

A variety of different genetic diseases in humans have been identified in which there is a defect in the peroxisomal alpha- and/or beta-oxidation of fatty acids, resulting in the accumulation of certain fatty acids in the plasma of these patients. See, Ferdinandusse, et al., J. Lipid Res. (2002) 43:438-444. Two groups can be distinguished. In the first group, both fatty acid alpha- and beta-oxidation are impaired. These patients, who suffer from a peroxisome biogenesis disorder, lack of functional peroxisomes and, as a consequence, are deficient for many processes taking place in the peroxisome, including the degradation of very long chain fatty acids and branched-chain fatty acids via beta-oxidation. In the second group, either the fatty acid alpha-oxidation or beta-oxidation is deficient, because in this group of patients only a single enzyme is deficient. Patients suffering from Refsum disease have deficiency of the first enzyme of the alpha-oxidation system (phytanoyl-CoA hydroxylase) and, as a consequence, accumulate phytanic acid in their plasma. Patients with a deficiency of D-bifunctional protein are deficient in peroxisomal beta-oxidation of both very long-chain fatty acids and the branched-chain fatty acids, including the bile acid intermediates. Ferdinandusse, et al., supra, recently discovered a new disorder, AMACR deficiency, which affects the peroxisomal oxidation of 2-methyl branched chain fatty acids and the bile acid intermediates. In AMACR deficiency, not only pristanic acid accumulates, but also one of the metabolites of pristanic acid, THA. Thus, AMACR is required for the complete degradation of pristanic acid.

In a screening assay in accordance with the present invention, the compound of interest is combined with cells, cell lysates, sub-cellular fractions, cell dispersion/suspension cell cultures from an individual and the combination is used as described above. The resulting material is analyzed by GC-MS (see, for example, Ferdinandusse, et al., and Reichel 2, both supra) to determine amounts of phytanic acid, pristanic acid and/or THA. The ratio of (2S,2R)-pristanic acid may be used to determine the amount of AMACR in the sample. The higher the level of 2R-pristanic acid, the lower is the amount of AMACR. The amount of AMACR is related to the effectiveness of the compound in the treatment of a disease. In the above system increases in the amount of AMACR indicate the increasing effectiveness of the compound of interest in the treatment of a disease.

It is also known that accumulation of (25R)-dihdroxycholestanoic acid (DHCA and (25R)-trihydroxycholestanoic acid (THCA) corresponds with AMACR deficiency. Accordingly, in a screening assay in accordance with the present invention, levels of (25R)-DHCA and (25R)-THCA may be measured by liquid chromatography-mass spectrometry (LC-MS) (see, for example, Ferdinandusse, et al., supra, and Ferdinandusse, et al., J. Lipid Res. (2001) 42:137-141) and related to the amount of AMACR, which in turn is influenced by the presence of a compound of interest. The less the amount of (25R)-DHCA and (25R)-THCA, the greater is the amount of AMACR. In the above system increases in the amount of AMACR indicate the increasing effectiveness of the compound of interest in the treatment of a disease.

The effectiveness of a compound of interest in the treatment of a disease is based on the ability of the compound to influence interactions involving AMACR. The compound of interest should have an influence on an interaction involving AMACR of at least about 10%, at least about 20%, at least about 30%, at least about 40%, at least about 50%, at least about 60%, at least about 70%, at least about 80%, at least about 90%, at least about 95%.

The present invention may be used alone to screen for compounds useful in the treatment of a disease or for the identification of a clinical/biological target for a disease. However, the present invention may also be used in conjunction with other assays for the identification of useful compounds. An assay in accordance with the present invention offers many advantages over known assays. The present methods permit one to screen a multitude of compounds and may be employed in conjunction with high throughput screening techniques whether known or developed in the future.

One particular example of a method in accordance with the present invention, by way of illustration and not limitation, is the screening of compounds, such as small organic compounds, using the enzymatic activity of AMACR. The example employs a standard assay for activity of AMACR as described by Shieh, et al., supra. In this example cancer cells known to over-express AMACR are homogenized by the addition of 2 volumes of lysis buffer 100 mM sodium phosphate pH 7.0, 1 mM phenylmethylsulfonyl fluoride, 3 mM EDTA, 0.25 M sucrose. The cell and lysis buffer mixture is homogenized using a Teflon glass tissue homogenizer for 5 passes at low speed. The lysate is centrifuged at 3,500×g to obtain the post-nuclear supernatant. This supernatant is used to assay for activity. The substrate for activity assay is S-2-(4-isobutylphenyl)propionyl-CoA (or S-ibuprofen CoA ester). The product of the reaction with AMACR, before work up, is R-2-(4-iosbutylphenyl)propionyl-CoA (R-ibuprofen CoA ester).

The reaction mixture contains 100 mM sodium phosphate pH 7.0, 1 mM EDTA, an appropriate amount of post-nuclear supernatant, 0.5 mM S-ibuprofen CoA ester, and the compound to be tested for AMACR effect in a total volume of 1 mL. The reaction is started by the addition of substrate and is shaken for 5 min. at 30° C. The reaction is stopped by addition of 0.3 mL of 2.5 M hydroxylamine pH 7.0. The hydroxamic acid is extracted with ether and then hydrolyzed with 6 M HCl. This provides the free acid of R- and/or S-ibuprofen. The HCl is extracted with water, and the ether layer dried. The amounts of R- and S-ibuprofen can be quantitated by chiral HPLC. The residue is dissolved in mobile phase (hexane:isopropanol:acetic acid; 98:2:0.5) and is chromatographed by HPLC on an S,S-Whelk O,1 column being eluted with mobile phase a 0.9 mL/min for 10 min using UV detection at 254 nm. The activity of the enzyme is expressed in μmoles of R-ibuprofen formed per min. at 30° C. A decrease in the amount of R-ibuprofen formed would indicate inhibition of AMACR by the test compound and an increase in the amount of R-ibuprofen produced would indicate a stimulation of AMACR.

Another specific example of a method in accordance with the present invention, by way of illustration and not limitation, is the screening of compounds, such as small organic compounds, to determine the effect on the amount/levels of AMACR. The methodology for doing this uses standard techniques adapted for this specific application. In this example cells in culture are treated with the compound to be screened and levels of AMACR are determined by Immunoblot (Western blot). Cell lines are maintained in culture in a humidified 5% CO₂ environment in DMEM (Dulbecco's Modified Eagle's Medium) plus 10% fetal calf serum (FCS). The compound to be tested is added to multiple cultures to achieve a wide concentration range including no addition of compound to be tested. The cells are incubated in the presence of the test compound for 24 hours. The cells are then lysed with Lysis buffer (1% Triton X-100, 50 mM Tris HCl, pH 7.5, 50 mM NaCl, 3 mM EDTA, 3 mM EGTA, 1 mM phenylmethylsulfonyl fluoride, 20 μg/mL aprotinin, 5 μg/mL leupepetin). The lysate containing 20 μg protein is mixed with SDS (sodium dodcecyl sulfate) sample buffer and separated by SDS-polyacrylamide gel electrophoresis in a 10% gel. The proteins are transferred to a nitrocellulose membrane. The membrane is blocked with Tris-buffered saline with 0.1% Tween 20 and 2% non-fat milk (2% Blocking Buffer) for 1 hour at room temperature. The membrane is then incubated with a rabbit antibody specific for AMACR in 2% Blocking Buffer at a dilution to interact with AMACR overnight at 4° C. The membrane is washed 3 times with Tris-buffered saline plus 0.1% TWEEN 20®. Anti-rabbit IgG-horseradish peroxidase conjugate in 2% Blocking Buffer is incubated with the membrane for 1 hour at room temperature. AMACR is visualized with the Amersham Enhanced Chemiluminescence system and autoradiography.

Kits for conducting any of the above methods can be employed. In the kit the reagents can be provided in packaged combination in the same or separate containers, depending on the cross-reactivity and stability of the reagents, so that the ratio of reagents provides for substantial optimization of a signal from the reporter molecule used in the detection system. The diagnostic kit can comprise an antibody specific for AMACR. This antibody may be attached to a reporter molecule. Alternatively, the kit can contain a second antibody that is attached to a reporter molecule. If necessary, the kit can also include other members of a signal producing system of which the reporter molecule is a part. Alternatively, the kit may include PCR primers, ligands for AMACR and so forth. Other reagents in the kit include ancillary agents such as buffering agents and protein stabilizing agents, and the like, calibrators, positive and negative control reagents, salts and so forth. The kit can also include supports or surfaces such as plates having wells for conducting methods in accordance with various aspects of the present invention. The supports or surfaces may be free of reagents or may contain one or more reagents bound thereto.

Another aspect of the present invention is a method for treating a disease comprising administering to a subject with the disease a pharmaceutically effective amount of a compound identified according to the aforementioned screening assay. Pharmaceutically effective amount is an amount of a compound administered to a subject in need thereof to achieve a preventative, therapeutic, curative or other effect thereby preventing the onset of, or preventing or alleviating the symptoms of, or causing the cure of, a disease.

The pharmaceutically effective amount of the above compound to be administered to a subject for treatment of a disease is dependent on the activity of the compound, the disease in question, the degree of symptoms, the body weight and age of the patient, the pathological stage of disease, family history, and disease associated risk factors and so forth. The dosage is readily determined by those skilled in the art based on the above factors. The doses can be administered in one large dose or several smaller doses on a daily basis.

The above compounds may be administered orally or parenterally (e.g., intramuscularly, subcutaneously, intravenously, rectally) in unit dose form or other such form. As a solid preparation, tablets, capsules, pills, granules, fine granules, powder and the like may be administered. As a semi-solid preparation, suppositories, transdermal systems, ointments and the like may be prepared. As a liquid preparation, injectable formulations, syrups, solutions, inhalants, emulsions, suspensions, suppositories, ovules, creams, emollients, etc. may be prepared.

Pharmaceutically acceptable additives and other materials typically used for such materials may also be included such as diluents, binders, disintegrators, lubricants, agents for delaying solubilization, coating materials, plasticizers, bases for suppositories, ointments, emulsions, flavors, sweeteners, and so forth.

These preparations can be modified to long-acting preparations or micro-capsules in any conventional manner. One or more kinds of the effective ingredients of the present invention may be generally contained at an amount of about 0.1 to about 99%, usually about 0.5 to about 90%, of the whole composition in the preparation.

In the preparations of the present invention, one or more other medicaments, for instance, anti-nociceptive (essentially anti-pain), anti-diabetics, anti-cholinesterases, anti-neoplastics and the like, may be used as combination therapy along with a compound identified in accordance with the present invention.

All publications and patents mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the present invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for screening a compound for use in the treatment of, or in the identification of a clinical or biological target for, a disease, said method comprising determining the ability of said compound to influence interactions involving alpha-methylacyl-CoA racemase.

2. A method according to claim 1 wherein said interactions involving alpha-methyacyl-CoA racemase comprise interaction of alpha-methylacyl-CoA racemase with a ligand for alpha-methyacyl-CoA racemase.

3. A method according to claim 1 wherein said interactions involving alpha-methylacyl-CoA racemase comprise an increase or decrease in an amount of alpha-methyacyl-CoA racemase.

4. A method according to claim 1 wherein said interactions involving alpha-methylacyl-CoA racemase comprise over-expression or under-expression of alpha-methylacyl-CoA racemase as a result of such disease.

5. A method according to claim 1 wherein said compound is selected from the group consisting of small organic compounds, proteins, carbohydrates and polynucleotides.

6. A method according to claim 1 wherein said ligand is a CoA thioester of an alpha-methyl acyl fatty acid where the alpha carbon is an R-stereoisomer.

7. A method according to claim 1 wherein said determining is carried out in vivo.

8. A method according to claim 1 wherein said determining is carried out in vitro.

9. A method according to claim 1 wherein said method comprises determining the amount or activity of alpha-methylacyl-CoA racemase resulting from the ability of said compound to influence the interaction of alpha-methyacyl-CoA racemase with a ligand for alpha-methylacyl-CoA racemase and relating the amount or activity thereof to the effectiveness of said compound in the treatment of, or in the identification of a clinical or biological target for, a disease.

10. A method according to claim 9 wherein said amount or activity of said alpha-methylacyl-CoA racemase is measured by measuring the amount of said ligand.

11. A method according to claim 1 wherein said method comprises determining the level of expression of alpha-methylacyl-CoA racemase resulting from the ability of said compound to influence the expression of alpha-methyacyl-CoA racemase and relating the level thereof to the effectiveness of said compound in the treatment of, or in the identification of a clinical or biological target for, a disease.

12. A method for screening a small organic compound for use in the treatment of a disease, said method comprising:
(a) forming an analysis system comprising said compound and alpha-methylacyl-CoA racemase,
(b) incubating said analysis system under conditions for an interaction involving said alpha-methylacyl-CoA racemase to occur, and
(c) measuring the amount or activity of alpha-methylacyl-CoA racemase in said system and relating the amount or activity thereof to the effectiveness of said compound in the treatment of a disease.

13. A method according to claim 12 wherein said analysis system comprises a host.

14. A method according to claim 13 wherein said host is selected from the group consisting of a mammal, a mammalian cell line and mammalian tissue.

15. A method according to claim 12 wherein said analysis system comprises a ligand for alpha-methylacyl-CoA racemase and said system is incubated under conditions for interactions involving said ligand and said alpha-methylacyl-CoA racemase to occur.

16. A method according to claim 15 wherein said ligand is a CoA thioester of an alpha-methyl acyl fatty acid where the alpha carbon is an R-stereoisomer.

17. A method according to claim 16 wherein said ligand is selected from the group consisting of branched chain fatty acids and C27 bile acid intermediate.

18. A method according to claim 16 wherein said fatty acid is phytanic acid, pristanic acid or trimethylundecanoic acid.

19. A method according to claim 15 wherein the amount or activity of said alpha-methylacyl-CoA racemase is measured by measuring the amount of said ligand.

20. A method for screening a small organic compound for use in the treatment of a disease, said method comprising:
(a) forming an analysis system comprising said compound, alpha-methylacyl-CoA racemase and a ligand for alpha-methylacyl-CoA racemase,
(c) incubating said analysis system under conditions for an interaction between said ligand and said alpha-methylacyl-CoA racemase to occur, and
(c) measuring the amount or activity of alpha-methylacyl-CoA racemase in said system and relating the amount or activity thereof to the effectiveness of said compound in the treatment of a disease.

21. A method according to claim 20 wherein the activity of said alpha-methylacyl-CoA racemase is measured.

22. A method according to claim 21 wherein the activity of said alpha-methylacyl-CoA racemase is measured by measuring the amount of said ligand.

23. A method for screening a small organic compound for use in the treatment of a disease, said method comprising:
(a) forming an analysis system comprising said compound and a host that expresses alpha-methylacyl-CoA racemase,
(d) incubating said analysis system under conditions for an interaction involving said alpha-methylacyl-CoA racemase to occur, and
(c) measuring the amount or activity of alpha-methylacyl-CoA racemase in said system and relating the amount or activity thereof to the effectiveness of said compound in the treatment of a disease.

24. A method according to claim 23 wherein said host is selected from the group consisting of a mammal, a mammalian cell line and mammalian tissue.

25. A method according to claim 23 wherein the amount or activity of said alpha-methylacyl-CoA racemase is measured by measuring the amount of alpha-methylacyl-CoA racemase mRNA or protein.

26. A method according to claim 23 wherein the amount or activity of said alpha-methylacyl-CoA racemase is measured by measuring the amount of polyA plus RNA.

27. A method according to claim 23 wherein the amount or activity of said alpha-methylacyl-CoA racemase is measured using an antibody specific for alpha-methylacyl-CoA racemase.

28. A method according to claim 23 wherein the amount or activity of said alpha-methylacyl-CoA racemase is measured by cDNA amplification.

29. A method according to claim 23 wherein the amount or activity of said alpha-methylacyl-CoA racemase is measured by measuring the amount of epimerase activity.

30. A method for treating a disease, said method comprising administering to a subject with said disease a pharmaceutically effective amount of a compound ascertained by the method of claim 1.

31. A method for treating a disease, said method comprising administering to a subject with said disease a pharmaceutically effective amount of a compound ascertained by the method of claim 12.

32. A method for treating a disease, said method comprising administering to a subject with said disease a pharmaceutically effective amount of a compound ascertained by the method of claim 20.

33. A method for treating a disease, said method comprising administering to a subject with said disease a pharmaceutically effective amount of a compound ascertained by the method of claim 23.
